(19) United States
(12) Patent Application Publication
(10) Pub. No.: US 2015/0057220 A1
(43) Pub. Date: Feb. 26, 2015

(54) FUSED AROMATIC PHOSPHONATE DERIVATIVES AS PRECURSORS TO PTP-1B INHIBITORS

(71) Applicant: KANEQ PHARMA INC., Boucherville (CA)

(72) Inventors: Michel Therien, Laval (CA); Yves Leblanc, Kirkland (CA); Yongxin Han, Montreal (CA)

(21) Appl. No.: 14/394,503

(22) PCT Filed: Apr. 16, 2013
(86) PCT No.: PCT/CA2013/000364

§ 371 Date: Oct. 15, 2014

Related U.S. Application Data
(60) Provisional application No. 61/624,572, filed on Apr. 16, 2012.

(51) Int. Cl. C07F 9/6571; A61K 45/06; A61K 31/662; A61K 31/675; A61K 31/665; C07F 9/40

(52) U.S. Cl. CPC C07F 9/657163; A61K 31/665; C07F 9/4021; A61K 31/662; A61K 31/675; A61K 45/06
USPC 514/6.5; 558/83; 514/110; 558/179; 514/112; 546/23; 514/82; 546/25; 514/89; 546/22; 514/11.7

(57) ABSTRACT

Fused aromatic phosphonates of structural formula I are precursors to inhibitors of protein tyrosine phosphatase-1B (PTP-1B). The compounds of the present invention are therefore useful for the treatment in a mammal of a disorder, condition, or disease responsive to inhibition of protein tyrosine phosphatase-1B, including Type 2 diabetes, insulin resistance, a lipid disorder, obesity, Metabolic Syndrome, and cancer.

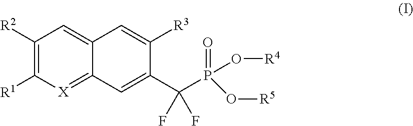

### CROSS-REFERENCE TO RELATED APPLICATIONS

This application claims priority of US provisional patent application 61/624,572, filed on Apr. 16, 2012, the specification of which is hereby incorporated by reference, in its entirety.

### FIELD OF THE INVENTION

The present invention is concerned with fused aromatic phosphonates, their synthesis, and their use as precursors to inhibitors of protein tyrosine phosphatase-1B (PTP-1B). The compounds of the present invention are precursors to inhibitors of PTP-1B and are therefore useful in the treatment of PTP-1B-mediated diseases, such as Type 2 diabetes, obesity, and cancer.

### BACKGROUND OF THE INVENTION

Protein tyrosine phosphatases are a large family of transmembrane or intracellular enzymes that dephosphorylate substrates involved in a variety of regulatory processes (Fischer et al., 1991, Science 253:401-406). Protein tyrosine phosphatase-1B (PTP-1B) is an approximately 50 kD intracellular protein present in abundant amounts in various human tissues (Charbonneau et al., 1989, Proc. Natl. Acad. Sci. USA 86:5252-5256; Goldstein, 1993, Receptor 3:1-15).

Numerous proteins are substrates of PTP-1B. One important substrate is the insulin receptor. The binding of insulin to its receptor results in autophosphorylation of the receptor, most notably on tyrosines 1146, 1150, and 1151 in the kinase catalytic domain (White & Kahn, 1994, J. Biol. Chem. 269:1-4). This causes activation of the insulin receptor tyrosine kinase, which phosphorylates the various insulin receptor substrate (IRS) proteins that propagate the insulin signaling event further downstream to mediate insulin's various biological effects.

Kennedy et al., 1999, Science 283: 1544-1548 showed that protein tyrosine phosphatase PTP-1B is a negative regulator of the insulin signalling pathway, suggesting that inhibitors of this enzyme may be beneficial in the treatment of Type 2 diabetes. Mice lacking PTP-1B are resistant to both diabetes and obesity.

Further support for the use of PTP-1B inhibitors to treat Type 2 diabetes and related diseases has been provided by the use of antisense oligonucleotides specific for PTP-1B in animal models of Type 2 diabetes. Inhibition of PTP-1B with antisense oligonucleotides in the animal models resulted in normalization of blood glucose and insulin levels. Zinker et al., 2002, Proc. Natl. Acad. Sci. USA, 99: 11357.

Compounds that inhibit PTP-1B are therefore expected to have utility for treating and/or controlling Type 2 diabetes and for improving glucose tolerance in patients in need thereof. Inhibitors of PTP-1B are also expected to be useful for delaying the onset of diabetes in pre-diabetic patients and for preventing pre-diabetic patients from developing diabetes. PTP-1B inhibitors may also have utility in treating obesity and dyslipidemia. A need therefore exists for novel chemical compounds that inhibit PTP-1B.

Elevated levels of PTP-1B have been observed in several cancer cell lines, including chronic myelogenous leukemia (CML), breast cancer, ovarian cancer, and prostate cancer, suggesting a regulatory role for PTP-1B in controlling kinase activity in these and other cancer cells. See for example, Liu, et al., J. Biol. Chem., 1996, 271:31290-31295; Kenneth et al., Mol Cell Biol, 1998, 18:2965-2975; Weiner et al., J. Natl. Cancer Inst., 1996, 86: 372-378. Thus inhibition of PTP-1B activity may constitute an important target for treating or preventing these and other cancers. PTP-1B inhibitors may thus be useful for treating or preventing cancer and for slowing the progression of cancer once it has developed.

Elevated levels of PTP-1B have also been detected by immunohistochemistry in various human cancers, including breast cancer, ovarian carcinomas, colon cancer, gastric cancer, squamous cell carcinomas and prostate cancer and this overexpression correlates with poor prognosis. See for example, Zhai et al., Cancer Res. 1993, 53: 2272-2278; Weiner et al., J. Natl. Cancer Inst.; Wiener, et al., Am. J. Obstet. Gynecol., 1994, 170: 1177-1183; Zhu et al., Cancer Res. 2007, 67; 10129-10137; Wang et al., Med. Oncol. 2011 Mar. 27. [Epub ahead of print; DOI: 10.1007s12032-011-9911-2]; Nanney et al., J. Cutan. Pathol., 1997, 24: 521-532; Wu et al., Prostate, 2006, 66: 1125-1135; Lessard et al., Cancer Res,. 2012 Jan. 26. [Epub ahead of print]. The overexperession of PTP-1B in human cancers and its correlation with tumor grade suggests that PTP-1B inhibitors may be useful in preventing the progression of these human cancers.

Julien et al, Nat. Genet., 2007, 39: 338-346, showed that NDL2 mice lacking one or two copies of the PTP-1B gene are tumor-free for a substantially longer period of time than those having normal copies of the gene. Furthermore, NDL2 mice treated with a PTP-1B inhibitor also show a significant delay in the formation of mammary tumors.

In addition, Balavenkatraman et. al., Mol Cancer Res., 2011, 9:1377-1384, demonstrated that PTP-1B activity contributes to human breast cancer onset which suggests that PTP1B inhibition may be effective in breast tumor prevention.

It is well-established that prodrugs may be used as a means of improving the physicochemical and pharmacokinetic properties of a drug molecule in order to improve its oral bioavailability. A prodrug moiety is then cleaved by a metabolic, enzymatic and/or chemical process in the body in order to generate the active moiety. Standard prodrugs consist of groups attached to a functionality on the drug [e.g. —OH, —SH, —COOH, —NH₂, —OP(O)(OH)₂, and —P(O)(OH)₂] that are cleaved from this functionality in vivo. Groups that are conventionally used to form prodrugs include, but are not limited to, carboxylic acid esters wherein the group is alkyl, aryl, acyloxyalkyl, or alkoxycarbonyloxyalkyl; acyl derivatives of hydroxyl, thiol and amines wherein the acyl group is alkylcarbonyl, alkoxycarbonyl, aminocarbonyl, phosphate or sulfate. Particular to this invention are groups that mask a phosphonic acid such as alkyl, aryl, acyloxyalkyl, and alkoxycarbonyloxyalkyl. Groups linked to the phosphorus atom via either an oxygen atom or a nitrogen atom may serve as prodrugs to the biologically active phosphonic acid. Since a phosphonic acid contains two functionalities that may be modified with prodrug groups, it is possible to have either one or two groups attached to the phosphorus atom through an oxygen atom. When two groups are attached, these two groups may be identical, may be two independent groups or may be linked together to form a ring which is itself a prodrug. In certain cases, multiple enzymatic, metabolic or chemical transformations may be required in order to convert the administered prodrug into the biologically active drug. Any stable intermediates generated in this stepwise process are also included in this invention.

Prodrug forms of biologically active compounds may have multiple utilities, for example, to improve oral bioavailability and thus allow for the administration of a smaller quantity of the medication; to improve palatability by masking or eliminating bitter taste or gastrointestinal irritability; to alter solubility to enable intravenous use; to provide for prolonged or sustained release or delivery of the biologically active compound; to improve ease of formulation; or to provide site-specific delivery of the biologically active compound. Commonly used prodrugs are described in (i) Ettmayer et al, J. Med. Chem. 2004, 47: 2393; (ii) Silverman, The Organic Chemistry of Drug Design and Drug Action, Academic Press, 1992, Chapter 8: “Prodrugs and Drug Delivery Systems: pg 352-401; (iii) Rautio et al, Nature Rev. Drug Disc. 2008, 7: 255. Additional examples of prodrugs of phosphonic acids are described in (i) Dang et al, J. Med. Chem. 2008, 51: 4331; (ii) Boutselis et al, J. Med. Chem. 2007, 50: 856; (iii) Farquhar et al, J. Med. Chem. 1994, 37: 3902; (iv) Lee et al, Antimicrob. Agents Chemother. 2005, 49: 1898; (v) Ballatore et al, Bioorg. Med. Chem. Lett. 2001, 11: 1053; (vi) Dang et al, J. Diabetes Met. 2010, 1: 105; (vii) Krise and Stella, Advanced Drug Deliv. Rev. 1996, 19: 287.

### SUMMARY OF THE INVENTION

The present invention relates to compounds of structural formula I:

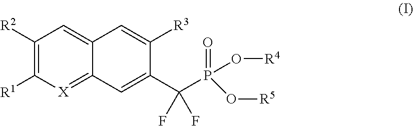

and pharmaceutically acceptable salt thereof; wherein

### X is CH or N;

R¹ is selected from the group consisting of (a) C₁₋₃ alkyl optionally substituted with 1-3 halogens, —OH, —OC₁₋₃ alkyl optionally substituted with 1-3 halogens, —SOₓC₁₋₃ alkyl, and —CN, (b) —CHO, (c) —(C═O)C₁₋₃ alkyl optionally substituted with 1-3 halogens, (d) —CN, (e) —(C═O)OC₁₋₃ alkyl optionally substituted with 1-3 halogens, (f) —(C═O)NHR⁶, (g) —CH═CH-aryl, (h) CH₂CH₂-aryl, (i) aryl, (j) heteroaryl, (k) —C≡C-aryl, and (l) —CH₂-aryl, wherein the —CH₂— group is optionally substituted with 1-2 substituents independently selected from halogen and C₁₋₂ alkyl optionally substituted with 1-3 halogens and wherein aryl and heteroaryl in all instances are optionally substituted with 1-3 substituents independently selected from (i) halogen, (ii) —(C═O)OC₁₋₃ alkyl optionally substituted with 1-3 halogens, (iii) —COOH, (iv) C₁₋₃ alkyl optionally substituted with 1-3 halogens, (v) OC₁₋₃ alkyl optionally substituted with 1-3 halogens, (vi) —SOₓMe, (vii) —CN, and (viii) —SO₂NH₂;
R² is selected from the group consisting of H, halogen, —CH₃, —CF₃, —OCH₃, and —OCF₃;
R³ is selected from the group consisting of H, halogen, and —OH;
R⁴ and R⁵ are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from (i) —(C═O)OR⁷, (ii) —(C═O)NHR⁷, (iii) —(C═O)N(R⁷)₂, (iv) —(C═O)NH₂, (v) —OR⁷, (vi) —O(C═O)R⁷, (vii) —O(C═O)OR⁷, (viii) —O(C—O)NHR⁷, (ix) —O(C═O)N(R⁷)₂, (x) —O(C═O)NH₂, (xi) —SO₂NH₂, (xii) —SOₓCH₃, (viii) —S(C═O)R⁷ and (ix) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
   or R⁴ and R⁵ together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl;
   with the provisos that (a) R⁴ and R⁵ cannot both be hydrogen, and (b) R⁴ or R⁵ cannot be C₁₋₃ alkyl optionally substituted with 1-3 halogens;
   R⁶ is selected from the group consisting of H, C₁₋₃ alkyl optionally substituted with 1-3 halogens, phenyl, or —CH₂-phenyl, wherein phenyl is optionally substituted with 1-3 substituents independently selected from (i) halogen, (ii) —(C═O)OC₁₋₃ alkyl optionally substituted with 1-3 halogens, (iii) —COOH, (iv) C₁₋₃ alkyl optionally substituted with 1-3 halogens, and (v) —OC₁₋₃ alkyl optionally substituted with 1-3 halogens;
   R⁷ is selected from the group consisting of C₁₋₆ alkyl optionally substituted with 1-3 substituents independently selected from (i) halogen, (ii) hydroxy, (iii)-OC₁₋₃ alkyl, (iv) aryl, and (v) heteroaryl, wherein aryl and heteroaryl are optionally substituted with 1-3 halogens, C₁₋₃ alkyl, C₁₋₃ haloalkyl, —CN, —SOCH₃, —SO₂NH₂, —COOH, and —OC₁₋₃ alkyl;
   R^{a} and R^{b} are each independently hydrogen or C₁₋₄ alkyl optionally substituted with hydroxy or 1-5 fluorines; and
   each x is independently an integer from 0 to 2.

The compounds of structural formula (I) are useful as precursors to phosphonic acid inhibitors of PTP-1B. Such compounds are therefore useful in the treatment of PTP-1B-mediated diseases, such as Type 2 diabetes and cancer.

Without limitation as to their mechanism of action, the fused aromatic phosphonate derivatives of the present invention act as precursors of the corresponding free phosphonic acids which have been demonstrated to be effective inhibitors of PTP-B. They are therefore useful for the treatment, control or prevention of disorders responsive to the inhibition of PTP-1B, such as Type 2 diabetes, insulin resistance, lipid disorders, obesity, atherosclerosis, Metabolic Syndrome and cancer.

Also encompassed within the present invention are pharmaceutical compositions comprising the compounds of formula (I) alone or in combination with other therapeutic agents active against the particular disease to be treated and a pharmaceutically acceptable carrier.

The present invention also relates to methods for the treatment, control, or prevention of disorders, diseases, or conditions responsive to inhibition of PTP-1B in a subject in need thereof by administering the compounds and pharmaceutical compositions of the present invention.

The present invention also relates to methods for the treatment, control, or prevention of Type 2 diabetes, insulin resistance, obesity, lipid disorders, atherosclerosis, Metabolic Syndrome and cancer by administering the compounds and pharmaceutical compositions of the present invention.

The present invention also relates to methods for the treatment, control, or prevention of obesity by administering the compounds of the present invention in combination with a therapeutically effective amount of one or more agents known to be useful to treat the condition.

The present invention also relates to methods for the treatment, control, or prevention of Type 2 diabetes by administering the compounds of the present invention in combination with a therapeutically effective amount of one or more agents known to be useful to treat the condition.

The present invention also relates to methods for the treatment, control, or prevention of atherosclerosis by administering the compounds of the present invention in combination with a therapeutically effective amount of one or more agents known to be useful to treat the condition.

The present invention also relates to methods for the treatment, control, or prevention of lipid disorders by administering the compounds of the present invention in combination with a therapeutically effective amount of one or more agents known to be useful to treat the condition.

The present invention also relates to methods for treating metabolic syndrome by administering the compounds of the present invention in combination with a therapeutically effective amount of one or more agents known to be useful to treat the condition.

The present invention also relates to methods for treating cancer by administering the compounds of the present invention in combination with a therapeutically effective amount of one or more agents known to be useful to treat the condition. Types of cancer that may be treated by compounds of the present invention include, but are not limited to, prostate cancer, breast cancer, ovarian cancer, multiple myeloma, leukemia, melanoma, lymphoma, gastric cancer, kidney cancer, bladder cancer, colon cancer and liver cancer.

### DETAILED DESCRIPTION OF THE INVENTION

The present invention relates to aromatic phosphonate compounds as precursors of aromatic phosphonic acid inhibitors of PTP-1B. Compounds of the present invention are described by structural formula I:

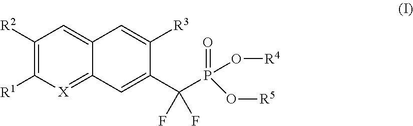

and pharmaceutically acceptable salt thereof; wherein

### X is CH or N;

R¹ is selected from the group consisting of (a) C₁₋₃ alkyl optionally substituted with 1-3 halogens, —OH, —OC₁₋₃ alkyl optionally substituted with 1-3 halogens, —SOₓC₁₋₃ alkyl, and —CN, (b) —CHO, (c) —(C═O)C₁₋₃ alkyl optionally substituted with 1-3 halogens, (d) —CN, (e) —(C═)OC₁₋₃ alkyl optionally substituted with 1-3 halogens, (f) —(C═O)NHR⁶, (g) —CH═CH-aryl, (h) —CH₂CH₂-aryl, (i) aryl, (j) heteroaryl, (k) —C≡C-aryl, and (l) —CH₂-aryl, wherein the —CH₂— group is optionally substituted with 1-2 substituents independently selected from halogen and C₁₋₂ alkyl optionally substituted with 1-3 halogens and wherein aryl and heteroaryl in all instances are optionally substituted with 1-3 substituents independently selected from (i) halogen, (ii) —(C—O)OC₁₋₃ alkyl optionally substituted with 1-3 halogens, (iii) —COOH, (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R² is selected from the group consisting of H, halogen, —CH₃, —CF₃, —OCH₃, and —OCF₃;
R³ is selected from the group consisting of H, halogen, and —OH;
R⁴ and R⁵ are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from (i) —(C═O)OR⁷, (ii) —(C—O)NHR⁷, (iii) —(C═O)N(R⁷)₂, (iv) —(C═O)NH₂, (v) —OR⁷, (vi) —O(C—O)R⁷, (vii) —O(C—O)OR⁷, (viii) —O(C—O)NHR⁷, (ix) —O(C═O)N(R⁷)₂, (x) —O(C—O)NH₂, (xi) —SO₂NH₂, (xii) —SOₓCH₃, (viii) —S(C═O)R⁷ and (ix) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
   or R⁴ and R⁵ together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl;
   with the provisos that (a) R⁴ and R⁵ cannot both be hydrogen, and (b) R⁴ or R⁵ cannot be C₁₋₃ alkyl optionally substituted with 1-3 halogens;
   R⁶ is selected from the group consisting of H, C₁₋₃ alkyl optionally substituted with 1-3 halogens, phenyl, or —CH₂-phenyl, wherein phenyl is optionally substituted with 1-3 substituents independently selected from (i) halogen, (ii) —(C═O)OC₁₋₃ alkyl optionally substituted with 1-3 halogens, (iii) —COOH, (iv) C₁₋₃ alkyl optionally substituted with 1-3 halogens, and (v) —OC₁₋₃ alkyl optionally substituted with 1-3 halogens;
   R⁷ is selected from the group consisting of C₁₋₆ alkyl optionally substituted with 1-3 substituents independently selected from (i) halogen, (ii) hydroxy, (iii) —OC₁₋₃ alkyl, (iv) aryl, and (v) heteroaryl, wherein aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃ alkyl;
   R^{a} and R^{b} are each independently hydrogen or C₁₋₄ alkyl optionally substituted with hydroxy or 1-5 fluorines; and
   each x is independently an integer from 0 to 2.

One embodiment of the current invention can be summarized by structural Formula Ia:

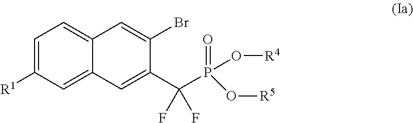

and pharmaceutically acceptable salts thereof, wherein:
R¹ is selected from the group consisting of (a) C₁₋₃ alkyl optionally substituted with 1-3 halogens or —CN, (b) —CHO, (c) —(C═O)C₁₋₃ alkyl optionally substituted with 1-3 halogens, (d) —CN, (e) —(C═O)NHR⁶, (f) —CH═CH-aryl, (g) aryl, (h) heteroaryl, (i) —C≡C-aryl, and (j) —CH₂-aryl, wherein the —CH₂— group is optionally substituted with 1-2 substituents independently selected from halogen and C₁₋₂ alkyl optionally substituted with 1-3 halogens and wherein aryl and heteroaryl in all instances are optionally substituted with 1-3 substituents independently selected from the group consisting of (i) halogen, (ii) —(C═O)OC₁₋₃ alkyl optionally substituted with 1-3 halogens, (iii) —COOH, (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R⁴ and R⁵ are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from (i) —(C═O)OR⁷, (ii) —(C═O)NHR⁷, (iii) —(C—O)N(R⁷)₂, (iv) —(C═O)NH₂, (v) —OR⁷, (vi) —O(C═O)R⁷, (vii) —O(C—O)OR⁷, (viii) —O(C—O)NHR⁷, (ix) —O(C═O)N(R⁷)₂, (x) —O(C═O)NH₂, (xi) —SO₂NH₂, (xii) —SOₓCH₃, (viii)-S(C═O)R⁷, and (xiii) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
   or R⁴ and R⁵ together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl;
   with the provisos that (a) R⁴ and R⁵ cannot both be hydrogen, and (b) R⁴ or R⁵ cannot be C₁₋₃ alkyl optionally substituted with 1-3 halogens;
   R⁶ is selected from the group consisting of H, C₁₋₃ alkyl optionally substituted with 1-3 halogens, phenyl, or —CH₂-phenyl, wherein phenyl is optionally substituted with 1-3 substituents independently selected from (i) halogen, (ii) —(C═O)OC₁₋₃ alkyl optionally substituted with 1-3 halogens, (iii) —COOH, (iv) C₁₋₃ alkyl optionally substituted with 1-3 halogens, and (v) —OC₁₋₃ alkyl optionally substituted with 1-3 halogens;
   R⁷ is selected from the group consisting of C₁₋₆ alkyl optionally substituted with 1-3 substituents independently selected from (i) halogen, (ii) —OC₁₋₃ alkyl, (iii) aryl, and (iv) heteroaryl, wherein wherein the aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃ alkyl;
   R^{a} and R^{b} are each independently hydrogen or C₁₋₄ alkyl optionally substituted with hydroxy or 1-5 fluorines; and
   each x is independently an integer from 0 to 2.

In a second embodiment of the compounds of structural formula (I) of the present invention, X is CH; R¹ is —CN or C₁₋₃ alkyl substituted with —CN; R² is hydrogen; and R³ is halogen. In a class of this embodiment, R¹ is —CN or —CH₂CN. In a subclass of this class, R¹ is —CH₂CN and R³ is bromine.

In a third embodiment of the compounds of structural formula (I) of the present invention, X is N; R¹ is —CN or C₁₋₃ alkyl substituted with —CN; R² is hydrogen; and R³ is halogen. In a class of this embodiment, R¹ is —CN or —CH₂CN. In a subclass of this class, R¹ is —CH₂CN and R³ is bromine.

In a fourth embodiment of the compounds of structural formula (I) of the present invention, R⁴ and R⁵ are each independently selected from aryl and heteroaryl wherein aryl and heteroaryl are optionally substituted with 1-3 halogens, C₁₋₃ alkyl, or C₁₋₃ haloalkyl. In a class of this embodiment, X is CH, R¹ is —CN or —CH₂CN, and R³ is bromine. In a second class of this embodiment, X is N, R¹ is —CN or —CH₂CN, and R³ is bromine.

In a fifth embodiment of the compounds of structural formula (I) of the present invention, R⁴ is hydrogen and R⁵ is aryl or heteroaryl wherein aryl and heteroaryl are optionally substituted with 1-3 halogens, C₁₋₃ alkyl, or C₁₋₃ haloalkyl. In a class of this embodiment, X is CH, R¹ is —CN or —CH₂CN, and R³ is bromine. In a second class of this embodiment, X is N, R¹ is —CN or —CH₂CN, and R³ is bromine.

In a sixth embodiment of the compounds of structural formula (I) of the present invention, R⁴ and R⁵ are each independently —(CR^{a}R^{b})₁₋₂ substituted with one substituent independently selected from (i) —O(C═O)R⁷, (ii) —O(C═O)OR⁷, (iii) —O(C—O)NHR⁷, (iv) —O(C═O)N(R⁷)₂, (v) —O(C═O)NH₂, and (vi) —S(C═O)R⁷ wherein R⁷, R^{a} and R^{b} are as described above. In a class of this embodiment, X is CH, R¹ is —CN or —CH₂CN, and R³ is bromine. In a second class of this embodiment, X is N, R¹ is —CN or —CH₂CN, and R³ is bromine. In a third class of this embodiment, R⁴ and R are each independently —(CR^{a}R^{b}) substituted with one substituent independently selected from (i) —O(C═O)R⁷, (ii) —O(C═O)OR⁷, (iii) —O(C—O)NHR⁷, (iv) —O(C═O)N(R⁷)₂, (v) —O(C═O)NH₂, and (vi) —S(C═O)R⁷. In a subclass of this third class, X is CH, R¹ is —CN or —CH₂CN, and R³ is bromine. In a second subclass of this third class, X is N, R¹ is —CN or —CH₂CN, and R³ is bromine.

In a seventh embodiment of the compounds of structural formula (I) of the present invention, R⁴ is hydrogen and R⁵ is —(CR^{a}R^{b})₁₋₂ substituted with one substituent independently selected from (i) —O(C—O)R⁷, (ii) —O(C—O)OR⁷, (iii) —O(C═O)NHR⁷, (iv) —O(C—O)N(R⁷)₂, (v) —O(C═O)NH₂, and (vi) —S(C═O)R⁷ wherein R⁷, R^{a} and R^{b} are as described above. In a class of this embodiment, X is CH, R¹ is —CN or —CH₂CN, and R³ is bromine. In a second class of this embodiment, X is N, R¹ is —CN or —CH₂CN, and R³ is bromine. In a third class of this embodiment, R⁵ is —(CR^{a}R^{b}) substituted with one substituent independently selected from (i) —O(C═O)R⁷, (ii) —O(C═O)OR⁷, (iii) —O(C═O)NHR⁷, (iv) —O(C═O)N(R⁷)₂, (v) —O(C═O)NH₂, and (vi) —S(C═O)R⁷. In a subclass of this third class, X is CH, R¹ is —CN or —CH₂CN, and R³ is bromine. In a second subclass of this third class, X is N, R¹ is —CN or —CH₂CN, and R³ is bromine.

In an eighth embodiment of the compounds of structural formula (I) of the present invention, R⁴ and R⁵ together with the phosphorus atom and the two oxygen atoms to which they are attached form a 6-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted by 1-3 halogens, C₁₋₃ alkyl, or C₁₋₃ haloalkyl. In a class of this embodiment, X is CH, R¹ is —CN or —CH₂CN, and R³ is bromine. In a second class of this embodiment, X is N, R¹ is —CN or —CH₂CN, and R³ is bromine. In a third class of this embodiment, the 6-membered ring is substituted with aryl or heteroaryl wherein aryl and heteroaryl are optionally substituted with 1-3 halogens, C₁₋₃ alkyl, or C₁₋₃ haloalkyl. In a subclass of this third class, X is CH, R¹ is —CN or —CH₂CN, and R³ is bromine. In a second subclass of this third class, X is N, R¹ is —CN or —CH₂CN, and R³ is bromine.

Illustrative, but nonlimiting, examples of compounds of the present invention that are useful as precursors of phosphonic acid inhibitors of PTP-1B are the following:

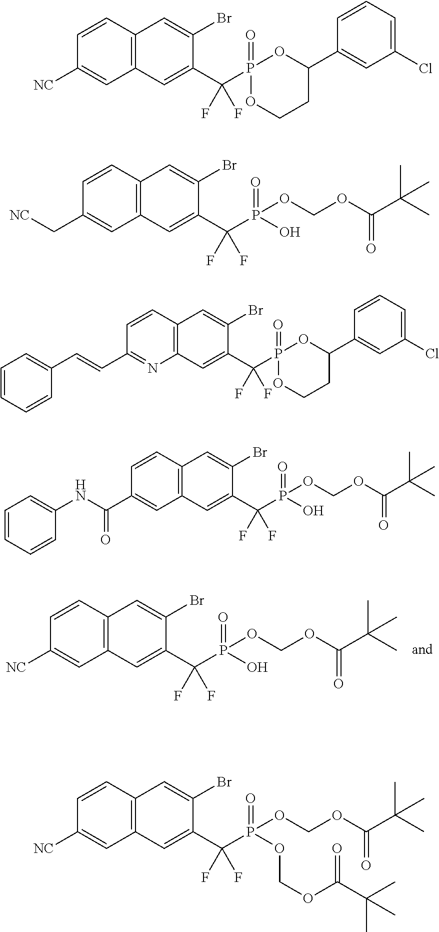 [SMILES: N#Cc1ccc2cc(Br)c(C(F)(F)P3(=O)OCCC(c4cccc(Cl)c4)O3)cc2c1 ; CC(C)(C)C(=O)OCOP(=O)(O)C(F)(F)c1cc2cc(CC#N)ccc2cc1Br ; O=P1(C(F)(F)c2cc3nc(/C=C/c4ccccc4)ccc3cc2Br)OCCC(c2cccc(Cl)c2)O1 ; CC(C)(C)C(=O)OCOP(=O)(O)C(F)(F)c1cc2cc(C(=O)Nc3ccccc3)ccc2cc1Br ; CC(C)(C)C(=O)OCOP(=O)(O)C(F)(F)c1cc2cc(C#N)ccc2cc1Br ; CC(C)(C)C(=O)OCOP(=O)(OCOC(=O)C(C)(C)C)C(F)(F)c1cc2cc(C#N)ccc2cc1Br]

and pharmaceutically acceptable salts thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus include heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

### Utilities

The compounds of the present invention of formula (I) are absorbed in the gastrointestinal track of a mammal and then converted by metabolic processes into the free phosphonic acid derivatives, which are known to be potent inhibitors of the PTP-1B enzyme. The conversion to an active inhibitor may be monitored by HPLC analysis of blood samples collected serially from the mammal following oral administration of a compound of the present invention. In some cases, the administered compound may be metabolically converted into one or more intermediate compounds which can be further metabolised into the active inhibitor of PTP-1B. In these cases, HPLC analysis of blood samples may indicate the presence of such intermediates as well as the active inhibitors of PTP-1B.

The administration of a compound of the present invention may provide a convenient and effective means of providing an efficacious concentration of the active free phosphonic acid PTP-1B inhibitor to a mammal that may benefit from inhibition of the FTP-1B enzyme. The active free phosphonic acid PTP-1B inhibitor may be prepared separately and shown in in vitro assays to effectively inhibit this enzyme. These active inhibitors generally have an IC₅₀ value of less than 1 μM in the enzyme assay described in the Assays section.

Inhibitors of PTP-1B improve insulin-sensitivity and may have utility in preventing or treating diabetes, improving glucose tolerance and insulin-sensitivity when there is insulin-resistance, and in treating or preventing obesity, all in mammals that are in need of such treatments or that may benefit from such treatments, including human beings. The compounds are more generally useful in treating Type 2 diabetes (non-insulin dependent diabetes, or NIDDM). The compounds may also cause a beneficial reduction in triglycerides and lipids.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating Metabolic Syndrome and its sequelae in a mammalian patient in need of such treatment comprising administering to said patient a compound in accordance with structural formula I in an amount that is effective to treat metabolic syndrome and its sequelae. The sequelae of the metabolic syndrome include hypertension, elevated blood glucose levels, high triglycerides, and low levels of HDL cholesterol.

A fifth aspect of the invention concerns a method of treating a lipid disorder selected from the group consisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the present invention concerns a method of treating other conditions that accompany Type 2 diabetes, including pancreatitis, adipose cell tumors, adipose cell carcinomas such as liposarcoma, inflammatory bowel disease, inflammation in general, and other disorders where insulin resistance is a component. By keeping hyperglycemia under control, the compounds may also be effective in delaying or preventing vascular restenosis and diabetic retinopathy.

An eighth aspect of the invention concerns a method of treating cancer in a mammalian patient in need of such treatment comprising administering to said patient a compound in accordance with structural formula I in an amount effective to treat cancer. Overexpression and elevated levels of PTP-1B have been observed in several cancer lines, including chronic myelogenous leukemia (CML), breast cancer, ovarian cancer, and prostate cancer, suggesting a regulatory role for PTP-1B in controlling kinase activity in these and other cancer cells. Thus inhibition of PTP-1B activity may constitute an important target for treating or preventing these and other cancers. The compounds may therefore be used to treat or prevent cancers, such as prostate cancer, breast cancer, ovarian cancer, multiple myeloma, leukemia, melanoma, lymphoma, renal cancer, gastric cancer and bladder cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) Metabolic Syndrome, (25) liver fibrosis, (26) cirrhosis of the liver, and (27) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) Metabolic Syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) Metabolic Syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting PTP-1B enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of cancer, hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of PTP-1B enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and/or “administering a” compound should be understood to mean providing a compound of the invention or a prodrug of a compound of the invention to the individual in need of treatment.

The utility of the compounds in accordance with the present invention as inhibitors of PTP-1B enzyme activity may be demonstrated by the following microsomal and whole-cell based assays:

### Assays for Measuring Biological Activity

Activity of the compounds of this application may be evaluated using the following assays for PTP-1B-inhibiting activity. As the claimed compounds are precursors of active phosphonic acid inhibitors, the compounds of this application will typically be inactive in this assay. In contrast, the corresponding phosphonic acid derivatives will have activities of less than 10 μM in this assay, and preferably, less than 1 μM.

### Enzyme Assay PTP-1B:

Assay buffer:
   50 mM Bis-Tris (pH=6.3)
   2 mM EDTA
   5 mM N,N′-dimethyl-N,N′-bis(mercaptoacetyl)hydrazine (DMH)
Substrate: 10 mM fluorescein diphosphate (FDP) store at −20° C. (also can use 10 mM DiFMUP)
Enzyme dilution buffer:
   50 mM Bis-Tris (pH=6.3)
   2 mM EDTA
   5 mM DMH
   20% (v/v) glycerol
   0.01% Triton X-100

The assay was carried out at room temperature in 96 well plates. The reaction mixture in 170 μl contained 50 mM Bis-Tris (pH=6.3), 2 mM EDTA, 5 mM N,N′-dimethyl-N,N′bis(mercaptoacetyl)hydrazine (DMH) and 10 μM fluorescein diphosphare (FDP) or 6,8-difluoro-4-methylumbelliferyl phosphate (DiFMUP). 10 μL of 10 concentrations (serial dilution) of the test compound (inhibitor) dissolved in DMSO or DMSO alone for control was added to each well and the plate was mixed for 2 min. The reaction was initiated by adding 20 μL of diluted PTP-1B (50 nM for FDP, 0.5 nM for DiFMUP in 50 mM Bis/Tris (pH=6.3), 2 mM EDTA, 5 mM DMH, 20% glycerol and 0.01% Triton X-100. The phosphatase activity was followed by monitoring the appearance of the fluorescent product fluorescein monophosphate (FMP) or 6,8-difluoro-7-hydroxyl-4-coumarin (DiFMU) continuously for 15-30 min, using the Spectromax Gemini fluorescent plate reader (Molecular probes) with excitation of 440 nm and emission at 530 nm (cutoff filter at 525 nm) for FDP and excitation at 360 nm and emission at 450 nm (cutoff filter at 435 nm) for DiFMUP. All the assays were done at least in duplicate. The initial rate of FMP or DiFMU formation is plotted against the concentration of inhibitor and the data are fitted to 4-parameter equation and the inflection point of the fit is the IC₅₀.

Assays for Measuring Oral Bioavailability of Compounds and their in Vivo Conversion into Active PTP-1B Inhibitors

### 1) Pharmacokinetics in Rats:

Per Os (PO) Pharmacokinetics in Rats

The animals are housed, fed and cared for according to the Guidelines of the Canadian Council on Animal Care.

Male Sprague Dawley rats (325-375 g) are fasted overnight prior to each study. The rats are placed in the restrainer one at a time and the box firmly secured. The baseline blood sample is obtained by nicking a small (1 mm or less) piece off the tip of the tail. The tail is then stroked with a firm but gentle motion from the top to the bottom to milk out the blood. Approximately 1 mL of blood is collected into a heparinized vacutainer tube.

Compounds are prepared as required, in a standard dosing volume of 10 mL/kg, and administered orally by passing a 16 gauge, 3″ gavaging needle into the stomach.

Subsequent bleeds are taken in the same manner as the baseline bleed except that there is no need to nick the tail again. The tail is cleaned with a piece of gauze and milked/stroked as described above into the appropriately labelled tubes.

Immediately after sampling, blood is centrifuged, separated, put into clearly marked vials and stored in a freezer until analysed.

Typical time points for determination of rat blood levels after PO dosing are 0, 15 min, 30 min, 1 h, 2 h, 4 h, 6 h, and 24 h.

After the 4 h time point bleed, food is provided to the rats ad libitum. Water is provided at all times during the study.

The following vehicles may be used in PO rat blood level determinations:
PEG 200/300/400: restricted to 2 mL/kg
Methocel 0.5%-1.0%:10 mL/g
Tween 80: 10 mL/kg

Compounds for PO blood levels can be in suspension form or in solution. For better dissolution or homogenous suspension, the solution can be placed in a sonicator for approximately 5 min.

For analysis, aliquots are diluted with an equal volume of acetonitrile and centrifuged to remove protein precipitate. The supernatant is injected directly onto a C-18 HPLC column with UV detection. Quantitation is done relative to a clean blood sample spiked with a known quantity of drug. Bioavailability (F) is assessed by comparing area under the curve (AUC) i.v. versus PO:

$$F = \frac{AUCpo}{AUCiv} \times \frac{DOSEiv}{DOSEpo} \times 100 \%$$

Clearance rates are calculated from the following relation:

$$CL = \frac{DOSEiv ( mg \text{/} kg )}{AUCiv}$$

The units of CL are mL/h·kg (milliliters per hour kilogram)

Intravenous (i.v.) Pharmacokinetics in Rats

The animals are housed, fed and cared for according to the Guidelines of the Canadian Council on Animal Care.

Male Sprague Dawley (325-375 g) rats are placed in plastic shoe box cages with a suspended floor, cage top, water bottle and food.

The compound is prepared as required, in a standard dosing volume of 1 mL/kg.

Rats are bled for the zero blood sample and dosed under CO₂ sedation. The rats, one at a time, are placed in a primed CO₂ chamber and taken out as soon as they have lost their righting reflex. The rat is then placed on a restraining board, a nose cone with CO₂ delivery is placed over the muzzle and the rat restrained to the board with elastics. With the use of forceps and scissors, the jugular vein is exposed and the zero sample taken, followed by a measured dose of compound which is injected into the jugular vein. Light digital pressure is applied to the injection site, and the nose cone is removed. The time is noted. This constitutes the zero time point.

The 5 min bleed is taken by nicking a piece (1-2 mm) off the tip of the tail. The tail is then stroked with a firm but gentle motion from the top of the tail to the bottom to milk the blood out of the tail. Approximately 1 mL of blood is collected into a heparinized collection vial. Subsequent bleeds are taken in the same fashion, except that there is no need to nick the tail again. The tail is cleaned with a piece of gauze and bled, as described above, into the appropriate labelled tubes.

Typical time points for determination of rat blood levels after I.V. dosing are either:
0, 5 min, 15 min, 30 min, 1 h, 2 h, and 6 h
or 0, 5 min, 30 min, 1 h, 2 h, 4 h, and 6 h.

### Vehicles:

The following vehicles may be used in IV rat blood level determinations:

Dextrose: 1 mL/kg

2-Hydroxypropyl-β-cyclodextrin 1 mL/kg

DMSO (dimethylsulfoxide): Restricted to a dose volume of 0.1 mL per animal

PEG 200: Not more than 60% mixed with 40% sterile water—1 mL/kg

With Dextrose, either sodium bicarbonate or sodium carbonate can be added if the solution is cloudy.

Determination of Bioavailability:

For analysis, aliquots are diluted with an equal volume of acetonitrile and centrifuged to remove protein precipitate. The supernatant is injected directly onto a C-18 HPLC column with UV or MS detection. Quantitation is done relative to a clean blood sample spiked with a known quantity of drug. Bioavailability (F) is assessed by comparing area under the curve (AUC) i.v. versus PO.

$$F = \frac{AUCpo}{AUCiv} \times \frac{DOSEiv}{DOSEpo} \times 100 \%$$

Clearance rates are calculated from the following relation:

$$CL = \frac{DOSEiv ( mg \text{/} kg )}{AUCiv}$$

The units of CL are mL/h·kg (milliliters per hour kilogram).

### 2) Pharmacokinetics in Mice

The animals are housed, fed and cared for according to the Guidelines of the Canadian Council on Animal Care. Pharmacokinetics were determined as described in Bateman et al, J Chromatoar B Biomed Sci Appl. 2001, 754: 245-51.

Per Os (PO) Pharmacokinetics in Mice

C57BL/6J mice are fasted overnight. A baseline bleed (0 h) is obtained by nicking a small piece off the tip of the tail. A small drop of blood is placed on an inverted weighing boat and a micropipette is used to accurately measure 10 μL of blood into a vial containing 30 μL of 0.1M trisodium citrate. The sample and buffer are aspirated several times in order to rinse all the blood from the pipette tip.

The animals are then dosed orally with the test compound in a suitable vehicle (usually 0.5% aqueous methocel) at a standard dose volume of 10 mL/kg by passing a gavaging needle into the stomach.

Subsequent bleeds are taken in the same manner as the baseline bleed except that there is no need to nick the tail again. The tail is cleaned with a piece of gauze and stroked to provide a fresh drop of blood to be sampled with a micropipette into trisodium citrate.

Each sample is diluted with 50 μL of acetonitrile containing a known concentration of an appropriate internal standard. Samples are vortexed to precipitate protein, then centrifuged. The supernatant is then analyzed by LCMS and compared to a standard curve of the test compound prepared in blank mouse blood, trisodium citrate and acetonitrile.

Intravenous (iv) Pharmacokinetics in Mice

This is carried out in the same manner as for oral dosing, except the dose of the test compound is injected into the jugular vein at a dose volume of 1 mL/kg in a suitable vehicle such as 0.9% saline solution, 5% aqueous dextrose solution, 25% aqueous 2-hydroxypropyl-b-cyclodextrin, or 60% aqueous PEG-200.

Determination of Bioavailability

Typical time points for determination of mouse blood levels after IV dosing are:
0, 5 min, 30 min, 1 h, 2 h, 6 h, and 24 h

Typical time points for determination of mouse blood levels after PO dosing are:
0, 15 min, 30 min, 1 h, 2 h, 6 h, and 24 h

Determination of blood concentrations at these timepoints can be used to generate a concentration vs time curve and an area under the curve (AUC) can be calculated.

Bioavailability (F) is assessed by comparing area under the curve (AUC) IV versus PO:

$$F = \frac{AUCpo}{AUCiv} \times \frac{DOSEiv}{DOSEpo} \times 100 \%$$

Clearance rates are calculated from the following relation:

$$CL = \frac{DOSEiv ( mg \text{/} kg )}{AUCiv}$$

The units of CL are mL/h·kg (milliliters per hour kilogram).

### 3) Oral Glucose Tolerance Test

Oral glucose tolerance tests are done on conscious Zucker obese fa/fa rats, obese ob/ob mice (age 12 weeks or older), or diet-induced obese (DIO) mice. The animals are fasted for 16-18 h before use for experiments. A test compound or a vehicle is given either intraperitoneally or orally 60 min before oral administration of a glucose solution at a dose of 2 g/kg body weight. Blood glucose levels are measured using a Medisense glucometer from tail bled samples taken at different time points before and after administration of glucose. A time curve of the blood glucose levels is generated and the area-under-the-curve (AUC) for 120 min is calculated (the time of glucose administration being time zero). Percent inhibition is determined using the AUC in the vehicle-control group as zero percent inhibition.

In separate studies, C57BL/6J mice are fed a high fat (35%) and high carbohydrate (36%) diet obtained from Bioserv (Frenchtown, N.J.) for 3 to 4 weeks, at which time the mice gained 50-100% of the baseline body weight. Oral glucose tolerance tests are done in the same manner as described above.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred, particularly in combination with a pharmaceutically acceptable carrier. However, the combination therapy may also include therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Examples of other active ingredients that may be administered in combination with a compound of Formula I, and either administered separately or in the same pharmaceutical composition, include, but are not limited to:

(1) dipeptidyl peptidase-IV (DPP-4) inhibitors;

(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), such as those disclosed in WO 02/060388, WO 02/08188, WO 2004/019869, WO 2004/020409, WO 2004/020408, and WO 2004/066963, and (4) PPARγ partial agonists; and (ii) biguanides, such as metformin and its pharmaceutically acceptable salts, in particular, metformin hydrochloride, and extended-release formulations thereof, such as Glumetza®, Fortamet®, and GlucophageXR®;

(3) insulin and insulin analogs or derivatives, such as insulin lispro, insulin detemir, insulin glargine, insulin glulisine, and inhalable formulations of each thereof;

(4) leptin and leptin derivatives, agonists, and analogs, such as metreleptin;

(5) amylin; amylin analogs, such as davalintide; and amylin agonists, such as pramlintide;

(6) sulfonylurea and non-sulfonylurea insulin secretagogues, such as tolbutamide, glyburide, glipizide, glimepiride, mitiglinide, and meglitinides, such as nateglinide and repaglinide;

(7) α-glucosidase inhibitors (such as acarbose, voglibose and miglitol);

(8) glucagon receptor antagonists, such as those disclosed in WO 98/04528, WO 99/01423, WO 00/39088, and WO 00/69810;

(9) incretin mimetics, such as GLP-1, GLP-1 analogs, derivatives, and mimetics (See for example, WO 2008/011446, U.S. Pat. No. 5,545,618, U.S. Pat. No. 6,191,102, and U.S. Pat. No. 56,583,111); and GLP-1 receptor agonists, such as oxyntomodulin and its analogs and derivatives (See for example, WO 2003/022304, WO 2006/134340, WO 2007/100535), glucagon and its analogs and derivatives (See for example, WO 2008/101017), exenatide, liraglutide, taspoglutide, albiglutide, AVE0010, CJC-1134-PC, NN9535, LY2189265, LY2428757, and BIM-51077, including intranasal, transdermal, and once-weekly formulations thereof, such as exenatide QW;

(10) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;

(11) HDL-raising drugs, such as niacin or a salt thereof and extended-release versions thereof; MK-524A, which is a combination of niacin extended-release and the DP-1 antagonist MK-524; and nicotinic acid receptor agonists;

(12) antiobesity compounds;

(13) agents intended for use in inflammatory conditions, such as aspirin, non-steroidal anti-inflammatory drugs (NSAIDs), glucocorticoids, and selective cyclooxygenase-2 (COX-2) inhibitors;

(14) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;

(15) glucokinase activators (GKAs), such as LY2599506;

(16) inhibitors of 11-hydroxysteroid dehydrogenase type 1, such as those disclosed in U.S. Pat. No. 6,730,690; WO 03/104207; and WO 04/058741;

(17) inhibitors of cholesteryl ester transfer protein (CETP), such as torcetrapib and MK-0859;

(18) inhibitors of fructose 1,6-bisphosphatase, such as those disclosed in U.S. Pat. Nos. 6,054,587; 6,110,903; 6,284,748; 6,399,782; and 6,489,476;

(19) inhibitors of acetyl CoA carboxylase-1 or 2 (ACC1 or ACC2);

(20) AMP-activated Protein Kinase (AMPK) activators;

(21) agonists of the G-protein-coupled receptors: GPR-109, GPR-116, GPR-119, and GPR-40, such as TAK-875, GW9508, and AMG 837;

(22) SSTR3 antagonists, such as those disclosed in WO 2009/011836;

(23) neuromedin U receptor 1 (NMUR1) and/or neuromedin U receptor 2 (NMUR2) agonists, such as those disclosed in WO2007/109135 and WO2009/042053, including, but not limited to, neuromedin U (NMU) and neuromedin S (NMS) and their analogs and derivatives;

(24) GPR-105 (P2YR14) antagonists, such as those disclosed in WO 2009/000087;

(25) inhibitors of glucose uptake, such as sodium-glucose transporter (SGLT) inhibitors and its various isoforms, such as SGLT-1; SGLT-2, such as dapagliflozin and remogliflozin; and SGLT-3;

(26) inhibitors of acyl coenzyme A:diacylglycerol acyltransferase 1 and 2 (DGAT-1 and DGAT-2);

(27) inhibitors of fatty acid synthase;

(28) inhibitors of acyl coenzyme A:monoacylglycerol acyltransferase 1 and 2 (MGAT-1 and MGAT-2);

(29) agonists of the TGR5 receptor (also known as GPBAR1, BG37, GPCR19, GPR131, and M-BAR);

(30) bromocriptine mesylate and rapid-release formulations thereof.;

(31) histamine H3 receptor agonists;

(32) α2-adrenergic or 3-adrenergic receptor agonists; and

(33) inhibitors of stearoyl Co-A desaturase-1 (SCD-1)

Dipeptidyl peptidase-IV (DPP-4) inhibitors that can be used in combination with compounds of Formula I include, but are not limited to, sitagliptin (disclosed in U.S. Pat. No. 6,699,871), vildagliptin, saxagliptin, alogliptin, denagliptin, carmegliptin, dutogliptin, melogliptin, linagliptin, SYR-472, and MK-472, and pharmaceutically acceptable salts thereof, and fixed-dose combinations of these compounds with immediate- or sustained-release metformin hydrochloride (such as JANUMET® and JANUMET XR®, and KOMBIGLYZE XR®), pioglitazone, rosiglitazone, simvastatin (JUVISYNC®), atorvastatin, or a sulfonylurea.

Other dipeptidyl peptidase-IV (DPP-4) inhibitors that can be used in combination with compounds of Formula I include, but are not limited to:
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-2-(2,5-difluorophenyl)tetrahydro)-5-(4,6-dihydropyrrolo[3,4-c]pyrazol-5(1H)-yl) tetrahydro-2H-pyran-3-amine;
(3R)-4-[(3R)-3-amino-4-(2,4,5-trifluorophenyl)butanoyl]-hexahydro-3-methyl-2H-1,4-diazepin-2-one;
4-[(3R)-3-amino-4-(2,5-difluorophenyl)butanoyl]hexahydro-1-methyl-2H-1,4-diazepin-2-one hydrochloride; and
(3R)-4-[(3R)-3-amino-4-(2,4,5-trifluorophenyl)butanoyl]-hexahydro-3-(2,2,2-trifluoroethyl)-2H-1,4-diazepin-2-one; and
   pharmaceutically acceptable salts thereof.

Antiobesity compounds that can be combined with compounds of Formula I include topiramate; zonisamide; naltrexone; phentermine; bupropion; the combination of bupropion and naltrexone; the combination of bupropion and zonisamide; the combination of topiramate and phentermine; fenfluramine; dexfenfluramine; sibutramine; lipase inhibitors, such as orlistat and cetilistat; melanocortin receptor agonists, in particular, melanocortin-4 receptor agonists; CCK-1 agonists; melanin-concentrating hormone (MCH) receptor antagonists; neuropeptide Y₁ or Y₅ antagonists (such as MK-0557); CB1 receptor inverse agonists and antagonists (such as rimonabant and taranabant); β₃ adrenergic receptor agonists; ghrelin antagonists; bombesin receptor agonists (such as bombesin receptor subtype-3 agonists); histamine H3 receptor inverse agonists; 5-hydroxytryptamine-2c (5-HT2c) agonists, such as lorcaserin; and inhibitors of fatty acid synthase (FAS). For a review of anti-obesity compounds that can be combined with compounds of the present invention, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); J. A. Fernandez-Lopez, et al., “Pharmacological Approaches for the Treatment of Obesity,” Drugs, 62: 915-944 (2002); and K. M. Gadde, et al., “Combination pharmaceutical therapies for obesity,” Exp. Opin. Pharmacother., 10: 921-925 (2009).

Glucagon receptor antagonists that can be used in combination with the compounds of Formula I include, but are not limited to:
N-[4-((1S)-1-{3-(3,5-dichlorophenyl)-5-[6-(trifluoromethoxy)-2-naphthyl]-1H-pyrazol-1-yl}ethyl)benzoyl]-β-alanine;
N-[4-((1R)-1-(3-(3,5-dichlorophenyl)-5-[6-(trifluoromethoxy)-2-naphthyl]-1H-pyrazol-1-yl)ethyl)benzoyl]-β-alanine;
N-(4-{1-[3-(2,5-dichlorophenyl)-5-(6-methoxy-2-naphthyl)-1H-pyrazol-1-yl]ethyl}benzoyl)-β-alanine;
N-(4-{(1S)-1-[3-(3,5-dichlorophenyl)-5-(6-methoxy-2-naphthyl)-1H-pyrazol-1-yl]ethyl}benzoyl)-N-alanine;
N-(4-{(1S)-1-[(R)-(4-chlorophenyl)(7-fluoro-5-methyl-1H-indol-3-yl)methyl]butyl}benzoyl)-β-alanine; and
N-(4-{(1S)-1-[(4-chlorophenyl)(6-chloro-8-methylquinolin-4-yl)methyl]butyl}benzoyl)-β-alanine; and
   pharmaceutically acceptable salts thereof.

Agonists of the GPR-119 receptor that can be used in combination with the compounds of Formula I include, but are not limited to:
rac-cis 5-chloro-2-{4-[2-(2-{[5-(methylsulfonyl)pyridin-2-yl]oxy}ethyl)cyclopropyl]piperidin-1-yl}pyrimidine;
5-chloro-2-{4-[(1R,2S)-2-(2-{[5-(methylsulfonyl)pyridin-2-yl]oxy}ethyl)cyclopropyl]piperidin-1-yl}pyrimidine;
rac cis-5-chloro-2-[4-(2-{2-[4-(methylsulfonyl)phenoxy]ethyl}cyclopropyl)piperidin-1-yl]pyrimidine;
5-chloro-2-[4-((1S,2R)-2-{2-[4-(methylsulfonyl)phenoxy]ethyl}cyclopropyl) piperidin-1-yl]pyrimidine;
5-chloro-2-[4-((1R,2S)-2-{2-[4-(methylsulfonyl)phenoxy]ethyl}cyclopropyl) piperidin-1-yl]pyrimidine;
rac cis-5-chloro-2-[4-(2-{2-[3-(methylsulfonyl)phenoxy]ethyl}cyclopropyl)piperidin-1-yl]pyrimidine; and
rac cis-5-chloro-2-[4-(2-{2-[3-(5-methyl-1,3,4-oxadiazol-2-yl)phenoxy]ethyl}cyclopropyl) piperidin-1-yl]pyrimidine; and
   pharmaceutically acceptable salts thereof.

Selective PPARγ modulators (SPPARγM's) that can be used in combination with the compounds of Formula I include, but are not limited to:
(2S)-2-({6-chloro-3-[6-(4-chlorophenoxy)-2-propylpyridin-3-yl]-1,2-benzisoxazol-5-yl}oxy)propanoic acid;
(2S)-2-({6-chloro-3-[6-(4-fluorophenoxy)-2-propylpyridin-3-yl]-1,2-benzisoxazol-5-yl}oxy)propanoic acid;
(2S)-2-{[6-chloro-3-(6-phenoxy-2-propylpyridin-3-yl)-1,2-benzisoxazol-5-yl]oxy}propanoic acid;
(2R)-2-({6-chloro-3-[6-(4-chlorophenoxy)-2-propylpyridin-3-yl]-1,2-benzisoxazol-5-yl}oxy)propanoic acid;
(2R)-2-{3-[3-(4-methoxy)benzoyl-2-methyl-6-(trifluoromethoxy)-1H-indol-1-yl]phenoxy}butanoic acid;
(2S)-2-{3-[3-(4-methoxy)benzoyl-2-methyl-6-(trifluoromethoxy)-1H-indol-1-yl]phenoxy}butanoic acid;
2-{3-[3-(4-methoxy)benzoyl-2-methyl-6-(trifluoromethoxy)-1H-indol-1-yl]phenoxy}-2-methylpropanoic acid; and
(2R)-2-{3-[3-(4-chloro)benzoyl-2-methyl-6-(trifluoromethoxy)-1H-indol-1-yl]phenoxy}propanoic acid; and
   pharmaceutically acceptable salts and esters thereof.

Inhibitors of 11β-hydroxysteroid dehydrogenase type 1 that can be used in combination with the compounds of Formula I include, but are not limited to:
3-[1-(4-chlorophenyl)-trans-3-fluorocyclobutyl]-4,5-dicyclopropyl-r-4H-1,2,4-triazole;
3-[1-(4-chlorophenyl)-trans-3-fluorocyclobutyl]-4-cyclopropyl-5-(1-methylcyclopropyl)-r-4H-1,2,4-triazole;
3-[1-(4-chlorophenyl)-trans-3-fluorocyclobutyl]-4-methyl-5-[2-(trifluoromethoxy)phenyl]-r-4H-1,2,4-triazole;
3-[1-(4-chlorophenyl)cyclobutyl]-4-methyl-5-[2-(trifluoromethyl)phenyl]-4H-1,2,4-triazole;
3-{4-[3-(ethylsulfonyl)propyl]bicyclo[2.2.2]oct-1-yl}-4-methyl-5-[2-(trifluoromethyl)phenyl]-4H-1,2,4-triazole;
4-methyl-3-{4-[4-(methylsulfonyl)phenyl]bicyclo[2.2.2]oct-1-yl}-5-[2-(trifluoromethyl)phenyl]-4H-1,2,4-triazole;
3-(4-{4-methyl-5-[2-(trifluoromethyl)phenyl]-4H-1,2,4-triazol-3-yl}bicyclo[2.2.2]oct-1-yl)-5-(3,3,3-trifluoropropyl)-1,2,4-oxadiazole;
3-(4-{4-methyl-5-[2-(trifluoromethyl)phenyl]-4H-1,2,4-triazol-3-yl}bicyclo[2.2.2]oct-1-yl)-5-(3,3,3-trifluoroethyl)-1,2,4-oxadiazole;
5-(3,3-difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
5-(1-fluoro-1-methylethyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
2-(1,1-difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole;
2-(3,3-difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and
5-(1,1-difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; and
   pharmaceutically acceptable salts thereof.

Somatostatin subtype receptor 3 (SSTR3) antagonists that can be used in combination with the compounds of Formula I include, but are not limited to:

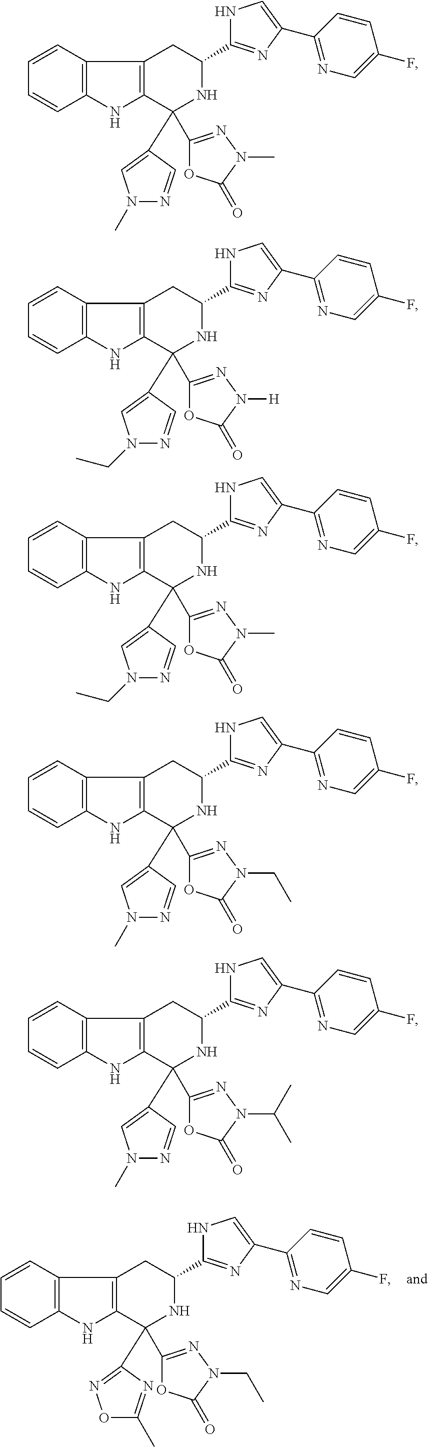

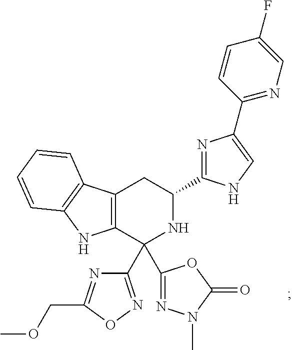 [SMILES: COCc1nc(C2(c3nn(C)c(=O)o3)N[C@@H](c3nc(-c4ccc(F)cn4)c[nH]3)Cc3c2[nH]c2ccccc32)no1]

and pharmaceutically acceptable salts thereof.

AMP-activated Protein Kinase (AMPK) activators that can be used in combination with the compounds of Formula I include, but are not limited to:

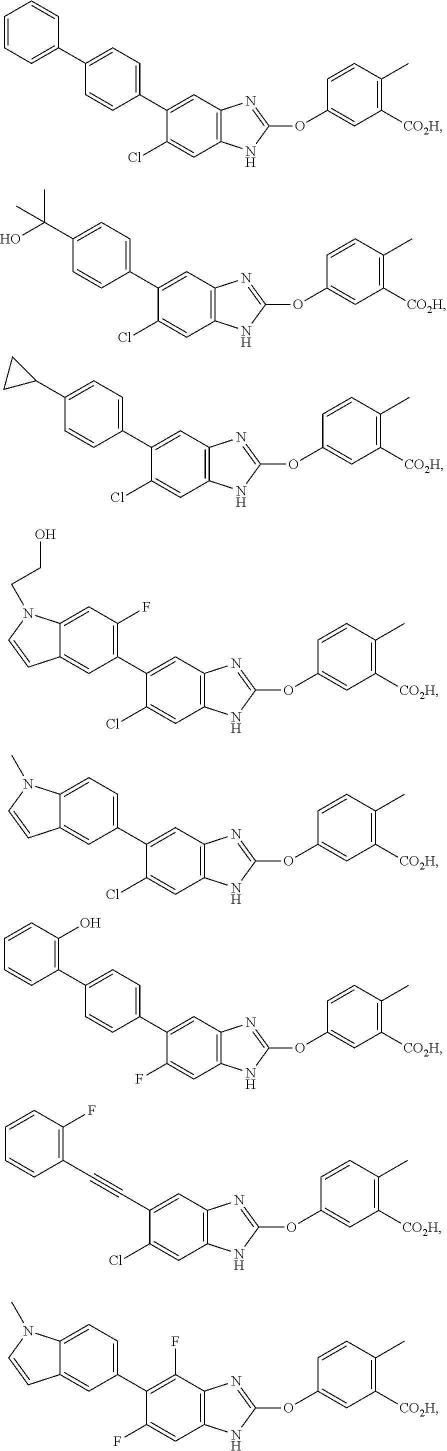

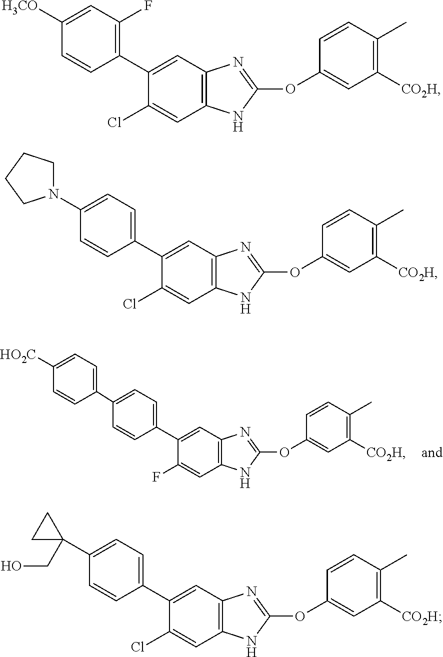 [SMILES: COc1ccc(-c2cc3nc(Oc4ccc(C)c(C(=O)O)c4)[nH]c3cc2Cl)c(F)c1 ; Cc1ccc(Oc2nc3cc(-c4ccc(N5CCCC5)cc4)c(Cl)cc3[nH]2)cc1C(=O)O ; Cc1ccc(Oc2nc3cc(-c4ccc(-c5ccc(C(=O)O)cc5)cc4)c(F)cc3[nH]2)cc1C(=O)O ; "and" ; Cc1ccc(Oc2nc3cc(-c4ccc(C5(CO)CC5)cc4)c(Cl)cc3[nH]2)cc1C(=O)O]

and pharmaceutically acceptable salts and esters thereof.

Inhibitors of acetyl-CoA carboxylase-1 and 2 (ACC-1 and ACC-2) that can be used in combination with the compounds of Formula I include, but are not limited to:
3-{1′-[(1-cyclopropyl-4-methoxy-1H-indol-6-yl)carbonyl]-4-oxospiro[chroman-2,4′-piperidin]-6-yl}benzoic acid;
5-{1′-[(1-cyclopropyl-4-methoxy-1H-indol-6-yl)carbonyl]-4-oxospiro[chroman-2,4′-piperidin]-6-yl}nicotinic acid;
1′-[(1-cyclopropyl-4-methoxy-1H-indol-6-yl)carbonyl]-6-(1H-tetrazol-5-yl)spiro[chroman-2,4′-piperidin]-4-one;
1′-[(1-cyclopropyl-4-ethoxy-3-methyl-1H-indol-6-yl)carbonyl]-6-(1H-tetrazol-5-yl)spiro[chroman-2,4′-piperidin]-4-one;
5-{1′-[(1-cyclopropyl-4-methoxy-3-methyl-1H-indol-6-yl)carbonyl]-4-oxo-spiro[chroman-2,4′-piperidin]-6-yl}nicotinic acid;
4′-({6-(5-carbamoylpyridin-2-yl)-4-oxospiro[chroman-2,4′-piperidin]-1′-yl}carbonyl)-2′,6′-diethoxybiphenyl-4-carboxylic acid;
2′,6′-diethoxy-4′-{[6-(1-methyl-1H-pyrazol-4-yl)-4-oxospiro[chroman-2,4′-piperidin]-1′-yl]carbonyl}biphenyl-4-carboxylic acid;
2′,6′-diethoxy-3-fluoro-4′-{([6-(-methyl-1H-pyrazol-4-yl)-4-oxospiro[chroman-2,4′-piperidin]-1′-yl]carbonyl}biphenyl-4-carboxylic acid;
5-[4-({6-(3-carbamoylphenyl)-4-oxospiro[chroman-2,4′-piperidin]-1′-yl}carbonyl)-2,6-diethoxyphenyl]nicotinic acid;
sodium 4′-({6-(5-carbamoylpyridin-2-yl)-4-oxospiro[chroman-2,4′-piperidin]-1′-yl}carbonyl)-2′,6′-diethoxybiphenyl-4-carboxylate;
methyl 4′-({6-(5-carbamoylpyridin-2-yl)-4-oxospiro[chroman-2,4′-piperidin]-1′-yl}carbonyl)-2′,6′-diethoxybiphenyl-4-carboxylate;
1′-[(4,8-dimethoxyquinolin-2-yl)carbonyl]-6-(1H-tetrazol-5-yl)spiro[chroman-2,4′-piperidin]-4-one;
(5-{1′-[(4,8-dimethoxyquinolin-2-yl)carbonyl]-4-oxospiro[chroman-2,4′-piperidin]-6-yl}-2H-tetrazol-2-yl)methyl pivalate;
5-{1′-[(8-cyclopropy-4-methoxyquinolin-2-yl)carbonyl]-4-oxospiro[chroman-2,4′-piperidin]-6-yl}nicotinic acid;
1′-(8-methoxy-4-morpholin-4-yl-2-naphthoyl)-6-(1H-tetrazol-5-yl)spiro[chroman-2,4′-piperidin]-4-one; and
1′-[(4-ethoxy-8-ethylquinolin-2-yl)carbonyl]-6-(1H-tetrazol-5-yl)spiro[chroman-2,4′-piperidin]-4-one; and
   pharmaceutically acceptable salts and esters thereof.

In another aspect of the invention, a pharmaceutical composition is disclosed which comprises:

(1) a compound of structural formula I;
(2) one or more compounds selected from the group consisting of:

(a) dipeptidyl peptidase IV (DPP-4) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; and (ii) biguanides, such as metformin and phenformin;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (NN-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib; and

(s) inhibitors of fructose 1,6-bisphosphatase, such as those disclosed in U.S. Pat. Nos. 6,054,587; 6,110,903; 6,284,748; 6,399,782; and 6,489,476; and

(t) agonists of GPR-40, such as TAK-875; and

(3) a pharmaceutically acceptable carrier.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients.

In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of PTP-1B enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing cancer, Type 2 diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Preparation of Compounds of the Invention

Synthetic methods for preparing the compounds of the present invention are illustrated in the following Schemes, Methods, and Examples. Starting materials are commercially available or may be prepared according to procedures known in the art or as illustrated herein. In some cases the order of carrying out the foregoing reaction schemes may be varied to facilitate the reaction or to avoid unwanted reaction products. The compounds of the invention are illustrated by means of the specific examples shown below. However, these specific examples are not to be construed as forming the only genus that is considered as the invention. These examples further illustrate details for the preparation of the compounds of the present invention. Those skilled in the art will readily understand that known variations of the conditions and processes of the following preparative procedures can be used to prepare these compounds. All temperatures are in degrees Celsius unless otherwise noted. Mass spectra (MS) were measured by electrospray ion-mass spectroscopy (ESI). ¹H NMR spectra were recorded on Bruker instruments at 400 or 500 MHz.

### LIST OF ABBREVIATIONS

Alk=alkyl
Ar=aryl
BINAP=2,2′-bis(diphenylphosphino)-1,1′-binaphthalene
Boc=tert-butoxycarbonyl
br=broad
CH₂Cl₂=dichloromethane
d=doublet
DBU=1,8-diazabicyclo[5.4.0]undec-7-ene
DEAD=diethyl azodicarboxylate

### DIPEA=N,N-diisopropylethylamine

DMF=dimethylformamide
DMSO=dimethyl sulfoxide
ESI=electrospray ionization
EtOAc=ethyl acetate
h=hours
HATU=O-(7-azabenzotriazol-1-yl)-N,N,N′,N′-tetramethyluronium hexafluorophosphate
HOAc=acetic acid
Hunig's base=N,N-diisopropylethylamine
LiOH=lithium hydroxide
m=multiplet
MeCN=acetonitrile
MeOH=methyl alcohol
MeTHF=2-methyltetrahydrofuran
MgSO₄=magnesium sulfate
min=minutes
MS=mass spectroscopy
MTBE=methyl tert-butyl ether
NaOH=sodium hydroxide
Na₂SO₄=sodium sulfate
NMP=N-methyl 2-pyrrolidinone
NMR=nuclear magnetic resonance spectroscopy
PG=protecting group
Ph=phenyl
rt=room temperature
s=singlet
t=triplet
TFA=trifluoroacetic acid
TFAA=trifluoroacetic anhydride
THF=tetrahydrofuran
TMEDA=N,N,N′,N′-tetramethylethylenediamine

### Method A:

A suitably substituted difluorophosphonic acid is converted to the corresponding phosphonyl chloride by treating with a chlorinating agent such as oxalyl chloride and catalytic DMF. The chloride atoms may then be displaced by an appropriate alcohol in the presence of a hindered amine base such as triethylamine or Hunig's base. If multiple equivalents of the alcohol are used, a bis-phosphonyl ester of the current invention is obtained directly. Otherwise, hydrolysis of the remaining chloride occurs on aqueous workup to give a monophosphonyl ester of the current invention. By adding two different alcohols, either sequentially or as a mixture, a mixed ester of the current invention is obtained.

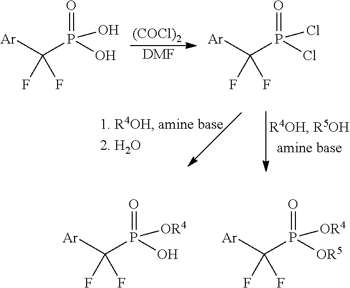

In a special case of Method A, if R⁴ and R⁵ are part of the same molecule, the resulting diol forms a cyclic phosphonate ester. Methods for preparing six-membered cyclic phosphonates are described in U.S. Pat. No. 6,312,662, the contents of which are herein incorporated by reference in their entirety.

### Method B:

A suitably substituted difluorophosphonic acid is treated with a suitable alkyl halide, such as chloride, bromide, and iodide, under basic conditions in a polar solvent such as DMF. This method works best for alkyl groups that have activated halide leaving groups due to the low nucleophilicity of the phosphonate anion. If one equivalent (eq.) of the alkyl halide is used, a mono-phosphonyl ester A of the current invention is obtained. If multiple equivalents of the alkyl halide are used, a bis-phosphonyl ester B of the current invention is obtained directly. By adding two different alkyl halides, either sequentially or as a mixture, a mixed ester C of the present invention is obtained.

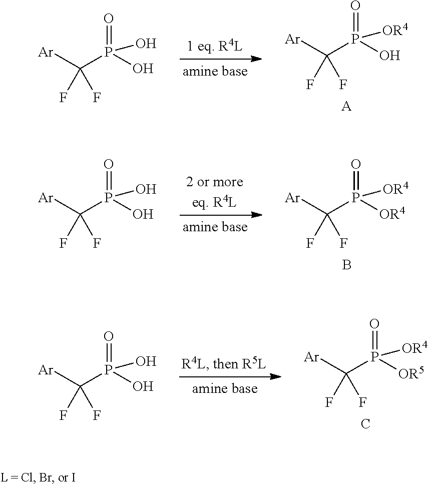

The following Examples are provided to illustrate the invention and are not to be construed as limiting the invention in any manner. The scope of the invention is defined by the appended claims.

### Example 1

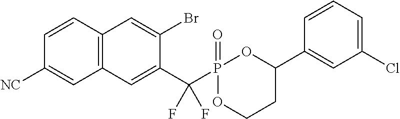 [SMILES: N#Cc1ccc2cc(Br)c(C(F)(F)P3(=O)OCCC(c4cccc(Cl)c4)O3)cc2c1]

To a solution of [(3-bromo-7-cyano-2-naphthyl)(difluoro)methyl]phosphonic acid (0.83 mmol) in dichloroethane (10 mL) was added DMF (0.08 mmol) and oxalyl chloride (6.6 mmol). The mixture was heated to 55° C. for 1.5 h, then concentrated. The residue was dissolved in dichloroethane (10 mL) and pyridine (1.7 mmol) was added. The resulting solution was transferred via cannula to a−78° C. solution of 1-(3-chlorophenyl)-1,3-propanediol (0.83 mmol) and N,N-diisopropylethylamine (5 mmol) in 1,2-dichloroethane (10 mL). The mixture was allowed to warm to room temperature and stirred for 1.5 h, then quenched with saturated aqueous NH₄Cl and extracted with EtOAc. The organic phase was washed with brine, dried over Na₂SO₄ and concentrated. Purification by silica gel chromatography gave 0.10 mmol of the desired compound.

¹H NMR (400 MHz, d₆-acetone) δ 8.68 (m, 1H), 8.50 (m, 2H), 8.18 (m, 1H), 7.93 (m, 1H), 7.58 (m, 1H), 7.5-7.4 (m, 3H), 6.12 (m, 1H), 5.0 (m, 1H), 4.75 (m, 1H), 2.57 (m, 1H), 2.46 (m, 1H).

### Example 2

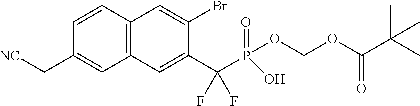 [SMILES: CC(C)(C)C(=O)OCOP(=O)(O)C(F)(F)c1cc2cc(CC#N)ccc2cc1Br]

To a solution of [3-bromo-7-(cyanomethyl)-2-naphthyl](difluoromethyl)phosphonic acid (0.33 mmol) in DMF (2.8 mL) was added chloromethylpivalate (0.83 mmol) and N,N-diisopropylethylamine (2.5 mmol). The mixture was heated to 60° C. overnight, then quenched with saturated aqueous NH₄Cl and extracted with EtOAc. The organic phase was washed with brine (3×), dried over Na₂SO₄ and concentrated. Purification by silica gel chromatography (2% HOAc/EtOAc) gave 0.10 mmol of the desired compound.

¹H NMR (400 MHz, d₆-acetone) δ 8.60 (m, 1H), 8.17 (m, 1H), 7.96 (m, 1H), 7.84 (m, 1H), 7.56 (m, 1H), 5.68 (d, 2H), 4.10 (s, 2H), 1.14 (s, 9H).

### Example 3

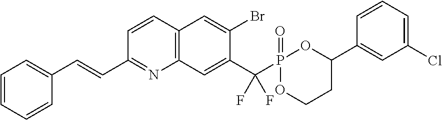 [SMILES: O=P1(C(F)(F)c2cc3nc(/C=C/c4ccccc4)ccc3cc2Br)OCCC(c2cccc(Cl)c2)O1]

Using the same procedure described for Example I, but starting with [(6-bromo-2-styrylquinolin-7-yl)(difluoro)methyl]phosphonic acid, the desired compound was obtained.

### Example 4

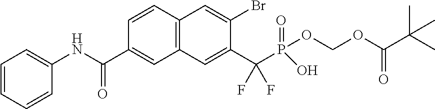 [SMILES: CC(C)(C)C(=O)OCOP(=O)(O)C(F)(F)c1cc2cc(C(=O)Nc3ccccc3)ccc2cc1Br]

Using the same procedure described for Example 2, but starting with [{2-[(phenylamino) carbonyl]-6-bromoquinolin-7-yl}(difluoro)methyl]phosphonic acid, the desired compound was obtained.

### Example 5

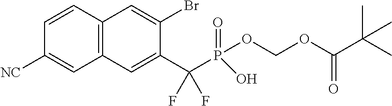 [SMILES: CC(C)(C)C(=O)OCOP(=O)(O)C(F)(F)c1cc2cc(C#N)ccc2cc1Br]

Using the same procedure described for Example 2, but starting with [(3-bromo-6-cyano-2-naphthyl)(difluoro)methyl]phosphonic acid, the desired compound was obtained.

### Example 6

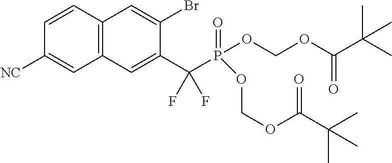 [SMILES: CC(C)(C)C(=O)OCOP(=O)(OCOC(=O)C(C)(C)C)C(F)(F)c1cc2cc(C#N)ccc2cc1Br]

Using the same procedure as in Example 5, but using 3 equivalents of chloromethylpivalolate and stirring at 55° C. overnight, the desired product was obtained.

The following additional compounds of structural formula (I) are prepared using the methods described above:

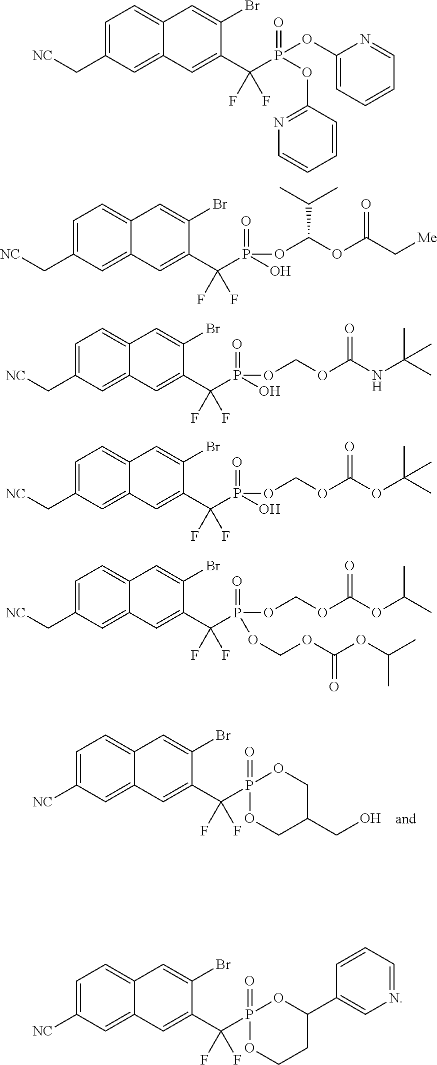

### Example 7

### Pharmacokinetic Data

The following compounds were administered orally to either mice or rats and blood samples analyzed for the corresponding phosphonic acid PTP— B inhibitor, showing that the prodrugs are converted into the active inhibitor in vivo.

| | | | Exposure of active |
|---|---|---|---|
| | Test | Dose | phosphonic acid |
| Example | species | administered | (Cmax) |
| 1 | mouse | 5 mg/kg PO | 1 μM |
| 2 | rat | 5 mg/kg PO | 14 μM |
| 2 | mouse | 5 mg/kg PO | 7 μM |
| 4 | mouse | 5 mg/kg PO | 3 μM |
| 5 | mouse | 5 mg/kg PO | 2.9 μM |
| 6 | mouse | 5 mg/kg PO | 3.3 μM |
| 6 | rat | 5 mg/kg PO | 1.1 μM |

### Example 8

### Efficacy in oGTT Assay

The compound of Example 2 was dosed orally in eDIO mice.

### Examples of Pharmaceutical Formulations

As a specific embodiment of an oral composition of a compound of the present invention, 50 mg of the compound of any of the Examples is formulated with sufficient finely divided lactose to provide a total amount of 580 to 590 mg to fill a size O hard gelatin capsule.

As a second specific embodiment of an oral pharmaceutical composition, a 100 mg potency tablet is composed of 100 mg of any one of the Examples, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 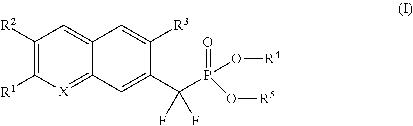
or a pharmaceutically acceptable salt thereof; wherein
X is CH or N;
R¹ is selected from the group consisting of (a) C₁₋₃ alkyl optionally substituted with 1-3 halogens, —OH, —OC₁₋₃ alkyl optionally substituted with 1-3 halogens, SOₓC₁₋₃ alkyl, and
—CN, (b) —CHO, (c) —(C═O)C₁₋₃ alkyl optionally substituted with 1-3 halogens, (d) —CN, (e) —(C═O)OC₁₋₃ alkyl optionally substituted with 1-3 halogens, (f) (C═O)NHR⁶, (g) —CH═CH-aryl, (h) —CH₂CH₂-aryl, (i) aryl, (j) heteroaryl, (k) —C≡C-aryl, and (l) —CH₂-aryl, wherein the —CH₂— group is optionally substituted with 1-2 substituents independently selected from halogen and C₁₋₂ alkyl optionally substituted with 1-3 halogens and wherein aryl and heteroaryl in all instances are optionally substituted with 1-3 substituents independently selected from (i) halogen, (ii) —(C═O)OC₁₋₃ alkyl optionally substituted with 1-3 halogens, (iii) —COOH, (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R² is selected from the group consisting of H, halogen, —CH₃, —CF₃, —OCH₃, and —OCF₃;
R³ is selected from the group consisting of H, halogen, and —OH;
R⁴ and R⁵ are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from (i) —(C═O)OR⁷, (ii) —(C═O)NHR⁷, (iii) —(C═O)N(R⁷)₂, (iv) —(C═O)NH₂, (v) —OR⁷, (vi) —O(C═O)R⁷, (vii) —O(C═O)OR⁷, (viii) —O(C═O)NHR⁷, (ix) —O(C═O)N(R⁷)₂, (x) —O(C═O)NH₂, (xi) —SO₂NH₂, (xii) —SOₓCH₃, (viii) —S(C═O)R⁷ and (ix) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
or R⁴ and R⁵ together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl;
with the proviso that R⁴ and R⁵ cannot both be hydrogen;
with the proviso that R⁴ and R⁵ cannot both be C₁₋₃ alkyl;
R⁶ is selected from the group consisting of H, C₁₋₃ alkyl optionally substituted with 1-3 halogens, phenyl, or —CH₂-phenyl, wherein phenyl is optionally substituted with 1-3 substituents independently selected from (i) halogen, (ii) —(C═O)OC₁₋₃ alkyl optionally substituted with 1-3 halogens, (iii) —COOH, (iv) C₁₋₃alkyl optionally substituted with 1-3 halogens, and (v) —OC₁₋₃ alkyl optionally substituted with 1-3 halogens;
R⁷ is selected from the group consisting of C₁₋₆ alkyl optionally substituted with 1-3 substituents independently selected from (i) halogen, (ii) hydroxy, (iii) —OC₁₋₃ alkyl, (iv) aryl, and (v) heteroaryl, wherein aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃alkyl;
R^{a} and R^{b} are each independently hydrogen or C₁₋₄ alkyl optionally substituted with hydroxy or 1-5 fluorines; and
each x is independently an integer from 0 to 2.

2. The compound of claim 1 of structural Formula Ia: 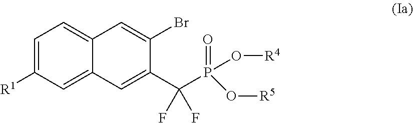
or a pharmaceutically acceptable salt thereof, wherein:
R¹ is selected from the group consisting of (a) C₁₋₃ alkyl optionally substituted with 1-3 halogens or —CN, (b) —CHO, (c) (C═O)C₁₋₃ alkyl optionally substituted with 1-3 halogens, (d) —CN, (e) (C═O)NHR⁶, (f) —CH═CH-aryl, (g) aryl, (h) heteroaryl, (i) —C≡C-aryl, and (j) —CH₂-aryl, wherein the —CH₂— group is optionally substituted with 1-2 substituents independently selected from halogen and C₁₋₂ alkyl optionally substituted with 1-3 halogens and wherein aryl and heteroaryl in all instances are optionally substituted with 1-3 substituents independently selected from the group consisting of (i) halogen, (ii) —(C═O)OC₁₋₃ alkyl optionally substituted with 1-3 halogens, (iii) —COOH, (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R⁴ and R⁵ are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from (i) —(C═O)OR⁷, (ii) —(C═O)NHR⁷, (iii) —(C═O)N(R⁷)₂, (iv) —(C═O)NH₂, (v) —OR⁷, (vi) —O(C═O)R⁷, (vii) —O(C═O)OR⁷, (viii) —O(C═O)NHR⁷, (ix) —O(C═O)N(R)₂, (x) —O(C═O)NH₂, (xi) —SO₂NH₂, (xii) —SOₓCH₃, (viii) —S(C═O)R⁷, and (xiii) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
or R⁴ and R⁵ together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl;
with the proviso that R⁴ and R⁵ cannot both be hydrogen;
with the proviso that R⁴ and R⁵ cannot both be C₁₋₃ alkyl;
R⁶ is selected from the group consisting of H, C₁₋₃ alkyl optionally substituted with 1-3 halogens, phenyl, or —CH₂-phenyl, wherein phenyl is optionally substituted with 1-3 substituents independently selected from (i) halogen, (ii) —(C═O)OC₁₋₃ alkyl optionally substituted with 1-3 halogens, (iii) —COOH, (iv) C₁₋₃ alkyl optionally substituted with 1-3 halogens, and (v) —OC₁₋₃ alkyl optionally substituted with 1-3 halogens;
R⁷ is selected from the group consisting of C₁₋₆ alkyl optionally substituted with 1-3 substituents independently selected from (i) halogen, (ii) —OC₁₋₃ alkyl, (iii) aryl, and (iv) heteroaryl, wherein the aryl and heteroaryl are optionally substituted with 1-3 halogens, C₁₋₃ alkyl, C₁₋₃ haloalkyl, —CN, SOₓCH₃, SO₂NH₂, —COOH, and —OC₁₋₃ alkyl;
R^{a} and R^{b} are each independently hydrogen or C₁₋₄ alkyl optionally substituted with hydroxy or 1-5 fluorines; and
each x is independently an integer from 0 to 2.

3. The compound of claim 1 or a pharmaceutically acceptable salt thereof, wherein X is CH; R¹ is —CN or —CH₂CN; and R³ is bromine.

4. (canceled)

5. The compound of claim 2 or a pharmaceutically acceptable salt thereof, wherein X is CH; R¹ is —CN or —CH₂CN and R³ is bromine.

6. The compound of claim 1 or a pharmaceutically acceptable salt thereof, wherein R⁴ and R⁵ are each independently selected from aryl and heteroaryl wherein aryl and heteroaryl are optionally substituted with 1-3 halogens, C₁₋₃ alkyl, or C₁₋₃ haloalkyl.

7. The compound of claim 6 or a pharmaceutically acceptable salt thereof, wherein X is CH, R¹ is —CN or —CH₂CN, and R³ is bromine.

8. The compound of claim 1 or a pharmaceutically acceptable salt thereof, wherein R⁴ is hydrogen and R⁵ is aryl or heteroaryl wherein aryl and heteroaryl are optionally substituted with 1-3 halogens, C₁₋₃ alkyl, or C₁₋₃ haloalkyl.

9. The compound of claim 8 or a pharmaceutically acceptable salt thereof, wherein X is CH, R¹ is —CN or —CH₂CN, and R³ is bromine.

10. The compound of claim 1 or a pharmaceutically acceptable salt thereof, wherein R⁴ and R⁵ are each independently —(CR^{a}R^{b})₁₋₂ substituted with one substituent independently selected from (i) —O(C═O)R⁷, (ii) —O(C═O)OR⁷, (iii) —O(C═O)NHR⁷, (iv) —O(C═O)N(R⁷)₂, (v) —O(C═O)NH₂, and (vi) —S(C═O)R⁷ wherein R⁷, R^{a} and R^{b} are as defined in claim 1.

11. The compound of claim 10 or a pharmaceutically acceptable salt thereof, wherein X is CH, R¹ is —CN or —CH₂CN, and R³ is bromine.

12. The compound of claim 1 or a pharmaceutically acceptable salt thereof, wherein R⁴ is hydrogen and R⁵ is —(CR^{a}R^{b})₁₋₂ substituted with one substituent independently selected from (i) —O(C═O)R⁷, (ii) —O(C═O)OR⁷, (iii) —O(C═O)NHR⁷, (iv) —O(C═O)N(R⁷)₂, (v) —O(C═O)NH₂, and (vi) —S(C═O)R⁷ wherein R⁷, R^{a} and R^{b} are as defined in claim 1.

13. The compound of claim 12 or a pharmaceutically acceptable salt thereof, wherein X is CH, R¹ is —CN or —CH₂CN, and R³ is bromine.

14. The compound of claim 1 or a pharmaceutically acceptable salt thereof, wherein R⁴ and R⁵ together with the phosphorus atom and the two oxygen atoms to which they are attached form a 6-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted by 1-3 halogens, C₁₋₃ alkyl, or C₁₋₃ haloalkyl.

15. The compound of claim 14 or a pharmaceutically acceptable salt thereof, wherein X is CH, R¹ is —CN or —CH₂CN, and R³ is bromine.

16. The compound of claim 1 selected from the group consisting of: 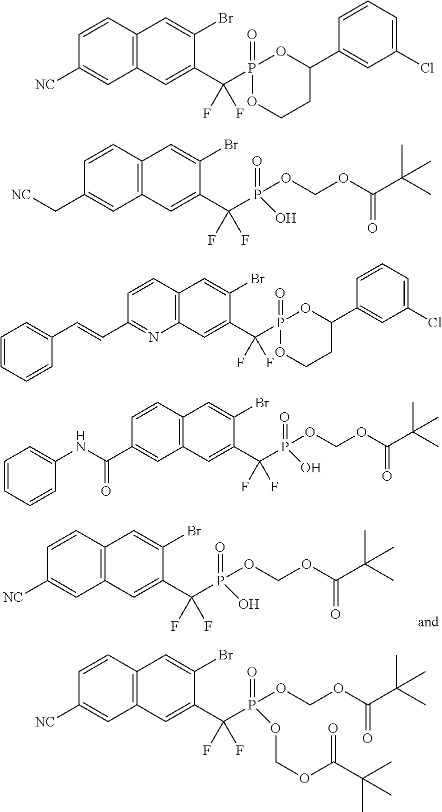
or pharmaceutically acceptable salt thereof.

17. A pharmaceutical composition comprising a compound in accordance with claim 1 or a pharmaceutically acceptable salt thereof, in combination with a pharmaceutically acceptable carrier.

18. A method for the treatment of Type 2 diabetes, insulin resistance, a lipid disorder, obesity, Metabolic Syndrome, and cancer comprising administering a compound according to claim 1 or a pharmaceutically acceptable salt thereof, to a mammal in need thereof.

19. The method according to claim 18, further comprising administering at least another therapeutically active compound selected from the group consisting of:
(a) PPAR gamma agonists and partial agonists;
(b) biguanides;
(c) GPR40 agonists;
(d) dipeptidyl peptidase IV (DP-IV) inhibitors;
(e) insulin or an insulin mimetic;
(f) sulfonylureas;
(g) α-glucosidase inhibitors;
(h) agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARa aqonists, (v) cholesterol absorption inhibitors, (h) acyl CoA:cholesterol acyltransferase (ACAT) inhibitors, (i) CETP inhibitors, and (i) phenolic anti-oxidants;
(i) PPARsα/γ dual agonists,
(j) PPARδ aqonists,
(k) antiobesity compounds,
(l) ileal bile acid transporter inhibitors;
(m) anti-inflammatory agents;
(n) glucagon receptor antagonists;
(o) GLP-1;
(p) GIP-1;
(q) GLP-1 analogs; and
(r) HSD-1 inhibitors.

20. The pharmaceutical composition according to claim 17, further comprising at least one other therapeutically active compound selected from the group consisting of
(a) PPAR gamma agonists and partial agonists;
(b) biguanides;
(c) GPR40 agonists;
(d) dipeptidyl peptidase IV (DP-IV) inhibitors;
(e) insulin or an insulin mimetic;
(f) sulfonylureas;
(g) α-glucosidase inhibitors;
(h) agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARa agonists, (v) cholesterol absorption inhibitors, (h) acyl CoA:cholesterol acyltransferase (ACAT) inhibitors, (i) CETP inhibitors, and (j) phenolic anti-oxidants;
(i) PPARα/γ dual agonists,
(j) PPARδ agonists,
(k) antiobesity compounds,
(l) ileal bile acid transporter inhibitors;
(m) anti-inflammatory agents;
(n) glucagon receptor antagonists;
(o) GLP-1;
(p) GIP-1;
(q) GLP-1 analogs; and
(r) HSD-1 inhibitors.

21. The compound of claim 1, wherein said compound of formula (I) is: 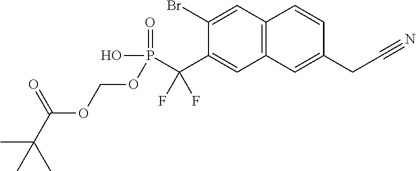 [SMILES: CC(C)(C)C(=O)OCOP(=O)(O)C(F)(F)c1cc2cc(CC#N)ccc2cc1Br]
or pharmaceutically acceptable salt thereof.

22. The composition of claim 20, wherein said compound of formula (I) is: 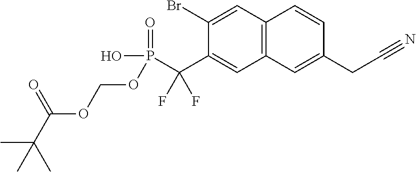 [SMILES: CC(C)(C)C(=O)OCOP(=O)(O)C(F)(F)c1cc2cc(CC#N)ccc2cc1Br]
or pharmaceutically acceptable salt thereof, and
said dipeptidyl peptidase IV (DP-IV) inhibitors is omarigliptin (MK-3102) or trelagliptin (SYR-472).
